(19) United States
(12) Patent Application Publication
(10) Pub. No.: US 2017/0084036 A1
(43) Pub. Date: Mar. 23, 2017

(54) REGISTRATION OF VIDEO CAMERA WITH MEDICAL IMAGING

(71) Applicant: Siemens Aktiengesellschaft, Munich (DE)

(72) Inventors: Thomas Pheiffer, Langhorne, PA (US); Stefan Kluckner, Princeton, NJ (US); Ali Kamen, Skillman, NJ (US)

(21) Appl. No.: 14/859,540

(22) Filed: Sep. 21, 2015

(51) Int. Cl. G06T 7/00; A61B 1/04; A61B 17/00; A61B 5/06; A61B 34/20; G06T 15/08; A61B 1/313

(52) U.S. Cl. CPC G06T 7/0038; G06T 7/003; G06T 7/0034; G06T 7/0075; G06T 15/08; A61B 1/04; A61B 1/3132; A61B 5/061; A61B 34/20; A61B 17/00234; G06T 2207/10016; G06T 2207/10068; G06T 2207/20212; A61B 2034/2055; A61B 2034/105

(57) ABSTRACT

Intraoperative camera data is registered with medical scan data. The same salient features are located in both the medical scan data and the model from the camera data. The features are specifically labeled rather than just being represented by the data. At least an initial rigid registration is performed using the salient features. The coordinate systems of the camera and the medical scan data are aligned without external positions sensors for the intraoperative camera.

### BACKGROUND

The present embodiments relate to medical imaging. In particular, camera images are registered with medical scan data.

The registration of videos to tomographic image volumes is an area of active research. Registration of endoscopic or laparoscopic video data to 3D image volumes is a challenging task due to intraoperative organ movements, which occur with phenomena like breathing or surgical manipulation. Due to the movement, correspondence between features in the video and features in the image volumes may be difficult to achieve.

In the domain of soft tissue interventions, registration is complicated by the presence of both rigid and non-rigid transformation components due to tissue deformation, which occurs over the course of the surgery. A typical strategy is to attach the intraoperative camera to an external tracking system, either optical or electromagnetic, in order to establish the absolute pose of the camera with respect to the patient. This tracker-based approach helps to establish an initial rigid registration between video and image volume, but introduces the burden of additional hardware requirements to the clinical workflow and the associated cost.

Other strategies rely only on the camera information in order to perform the registration. A patient-specific 3D model of the organ of interest is created by stitching together sequences of 2D or 2.5D images or video from the camera. This intraoperative reconstructed model may then be fused with preoperative or intraoperative volumetric data to provide additional guidance to the clinician. The registration is challenging to compute in practice due to a lack of constraints on the problem and the very different natures of the 3D model and the volumetric data.

### BRIEF SUMMARY

By way of introduction, the preferred embodiments described below include methods, systems, instructions, and computer readable media for registration of intraoperative camera data with medical scan data. The same salient features are located in both the medical scan data and the model from the camera data. The features are specifically labeled rather than just being represented by the data. At least an initial rigid registration is performed using the salient features. The coordinate systems of the camera and the medical scan data are aligned without external positions sensors for the intraoperative camera.

In a first aspect, a method is provided for registration of a video camera with a preoperative volume. An atlas labeled with first salient features is fit to the preoperative volume of a patient. Depth measurements are acquired from an endoscope or laparoscope having the video camera and inserted within the patient. A medical instrument in the patient is imaged with the video camera. Indications of second salient features are received by the medical instrument being positioned relative to the second salient features. A three-dimensional distribution of the depth measurements labeled with the second salient features is created. The three-dimensional distribution is registered with the preoperative volume using the second salient features of the three-dimensional distribution and the first salient features of the preoperative volume. An image of the patient is generated from the preoperative volume and a capture from the video camera. The image is based on the registering of the preoperative volume with the three-dimensional distribution.

In a second aspect, a non-transitory computer readable storage medium has stored therein data representing instructions executable by a programmed processor for registration with medical scan data. The storage medium includes instructions for identifying salient features in the medical scan data representing a patient, the medical scan data being from a medical scanner, identifying the salient features in video images from an intraoperative camera and positioning of a tool within the patient, and registering coordinates systems of the medical scan data from the medical scanner with the intraoperative camera using the identified salient features.

In a third aspect, a system is provided for registration. An intraoperative camera is operable to captures images from within a patient. A minimally invasive surgical tool is operable to be inserted into the patient. A memory is configured to store data representing labeled anatomy of the patient, the data being from a medical imager. A processor is configured to locate anatomical positions using the surgical tool represented in the images and to register the images with the data using the labeled anatomy and the anatomical positions.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flow chart diagram of one embodiment of a method for registration of a video camera with a preoperative volume;

FIG. 2 illustrates an example of a method for registration of intraoperative information with scan data; and

FIG. 3 is one embodiment of a system for registration.

### DETAILED DESCRIPTION OF THE DRAWINGS AND PRESENTLY PREFERRED EMBODIMENTS

3D endoscopic or laparoscopic video is registered to preoperative or other medical imaging using salient features. For registration, the additional or alternative correspondences in the form of anatomical salient features are used. A statistical atlas of organ features are mapped to the preoperative image or scan data for a specific patient in order to facilitate registration of that data to organ features digitized intraoperatively by tracking surgical instruments in the endoscopic video. The weighted registration matches the salient features in the two data sets (e.g., intraoperative and preoperative).

In one embodiment, the tip of a surgical tool is tracked in the intraoperative endoscopic video. By placement of the tool relative to salient features, the tool and tracking in the coordinate system of the video is used to digitize a set of salient features on the organ or in the patient that correspond to a set of known features in the preoperative imaging. An external optical tracking system to track the surgical instrument may not be needed.

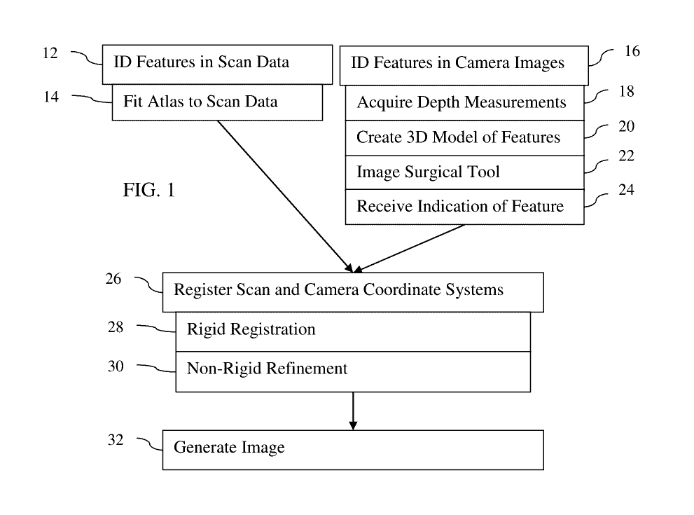

FIG. 1 shows a flow chart of one embodiment of a method for registration of a video camera with a medical scan volume. For example, endoscopic or laparoscopic video images are registered with preoperative or intraoperative 3D image volumes. The registration is guided by establishing correspondence between salient features identified in each modality.

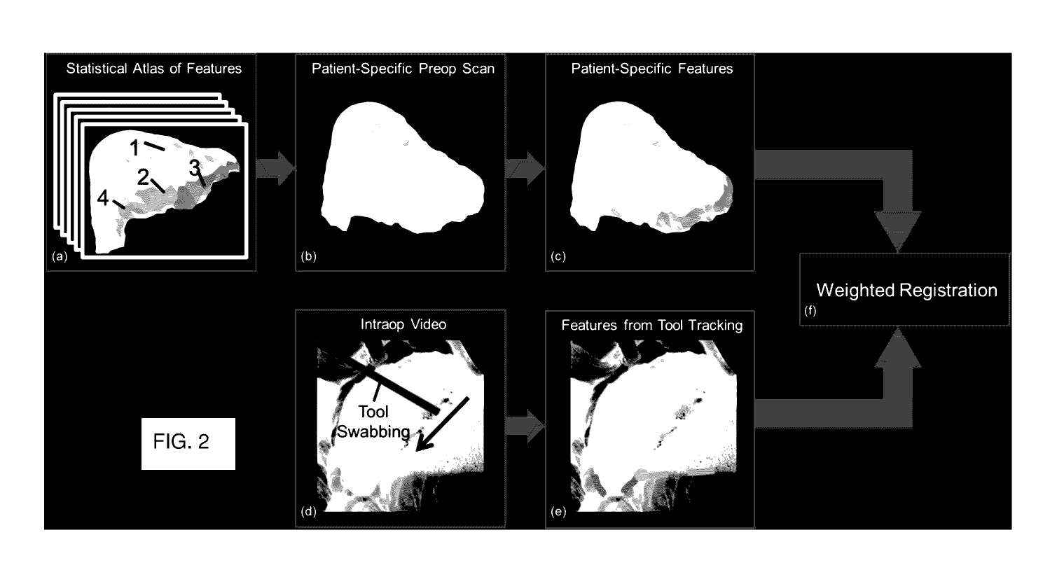

FIG. 2 shows another embodiment of the method. A 3D tomographic image volume and a sequence of 2D laparoscopic or endoscopic images with 2.5D depth data are used. The preoperative image is processed by fitting with an atlas including feature labels. Through interaction with intraoperative video, feature labels are provided for a 3D model from the 2.5D depth data. The features from the image volume and the 3D model are rigidly registered, providing a transform that at least initially aligns the two image datasets to each other.

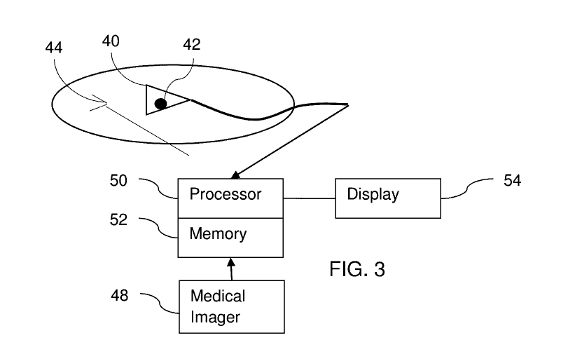

The methods are implemented by the system of FIG. 3 or another system. For example, some acts of one of the methods are implemented on a computer or processor associated with or part of a computed tomography (CT), magnetic resonance (MR), positron emission tomography (PET), ultrasound, single photon emission computed tomography (SPECT), x-ray, angiography, or fluoroscopy imaging system. As another example, the method is implemented on a picture archiving and communications system (PACS) workstation or implemented by a server. Other acts use interaction with other devices, such as the camera and/or surgical tool, for automated or semi-automated feature labeling and/or registration.

The acts are performed in the order shown or other orders. For example, act 12 is performed prior to, simultaneously, or after act 16. Any of the acts 14 for implementing act 12 and acts 18-24 for implementing act 16 may be interleaved or performed prior to or after each other. In one embodiment, acts 18 and 20 are performed simultaneously, such as where the camera-captured images are used to determine the depth, but may be performed in any order.

Additional, different, or fewer acts may be provided. For example, the method is performed using acts 12, 16, and/or 26, but with different sub-acts (e.g., 14, and 18-24) to identify the features in the scan data and/or the camera images and/or sub-acts (28-30) to register. As another example, act 32 is not provided, but instead the registration is used to control or provide other feedback.

In act 12, features are identified in scan data. Any type of scan data may be used. A medical scanner, such as a CT, x-ray, MR, ultrasound, PET, SPECT, fluoroscopy, angiography, or other scanner provides scan data representing a patient. The scan data is output by the medical scanner for processing and/or loaded from a memory storing a previously acquired scan.

The scan data is preoperative data. For example, the scan data is acquired by scanning the patient before the beginning of a surgery, such as a minutes, hours, or days before. Alternatively, the scan data is from an intraoperative scan, such as scanning while minimally invasive surgery is occurring.

The scan data, or medical imaging data, is a frame of data representing the patient. The data may be in any format. While the term “image” is used, the image may be in a format prior to actual display of the image. For example, the medical image may be a plurality of scalar values representing different locations in a Cartesian or polar coordinate format the same as or different than a display format. As another example, the medical image may be a plurality red, green, blue (e.g., RGB) values to be output to a display for generating the image in the display format. The medical image may be currently or previously displayed image in the display format or other format.

The scan data represents a volume of the patient. The patient volume includes all or parts of the patient. The volume and corresponding scan data represent a three-dimensional region rather than just a point, line or plane. For example, the scan data is reconstructed on a three-dimensional grid in a Cartesian format (e.g., NxMxR grid where N, M, and R are integers greater than one). Voxels or other representation of the volume may be used. The scan data or scalars represent anatomy or biological activity, so is anatomical and/or functional data.

The volume includes one or more features. The scan data represents the salient features, but without labeling of the salient features. The features are salient features, such as anatomical features distinguishable from other anatomy. In a liver example, the features may be ligaments and/or ridges. The features may be a point, line, curve, surface, or other shape. Rather than entire organ surfaces associated with segmentation, the surface or other features are more localized, such as a patch less than 25% of the entire surface. Larger features, such as the entire organ surface, may be used. Alternatively or additionally, the features are functional features, such as locations of increased biological activity.

The features are identified in the medical scan data. Rather than just representing the features, the locations of the features are determined and labeled as such. In one embodiment, one or more classifiers identify the features. For example, machine-learnt classifiers, applied by a processor, identify the location or locations of the features.

In another embodiment, an atlas is used in act 14. To automate the assignment of salient features to the patient-specific scan data, an atlas is used. The atlas includes the features with labels for the features. The atlas represents the organ or organs of interest. For example, a statistical atlas is constructed by annotating the salient features in a large set of images or volumes from many patients whom are representative of the population undergoing the intervention. The atlas is the result of an analysis of these data, such as with machine and/or deep learning algorithms.

The atlas is registered with the scan data so that the labeled features of the generic atlas are transformed to the patient. The locations of the features in the scan data are located by transforming the labeled atlas to the scan data. FIG. 2 represents this where (a) shows an atlas of features to be registered with a preoperative scan (b) of the same anatomy. After registration, the labels from the atlas are provided (c) for the voxels of the scan data. This registration to identify the features in the scan data only needs to be performed once, although the atlas may be expanded with additional patient images over the course of time and the fitting performed again for the same patient.

Any fitting of the statistical atlas or other model to the medical scan data may be used. The fitting is non-rigid or affine, but may be rigid in other embodiments. A processor registers the atlas to the preoperative image or other volume for that patient. Any now known or later developed registration may be used. For example, a 3D-3D registration is performed with flows of diffeomorphisms. Once the atlas is registered, the patient-specific salient feature locations in the preoperative image volume become known as shown in FIG. 2c.

Referring again to FIG. 1, a processor identifies the features in the video images from an intraoperative camera and positioning of a tool within the patient. The pose of a surgical instrument is tracked intraoperatively in the video data and is used to digitize salient features. The intraoperative data includes a video stream or captured image from a minimally invasive camera system, such as an endoscope or laparoscope. The images captured by the camera and/or depth data from a separate sensor may be used to reconstruct a 3D surface of the scene. The 3D surface or model of the patient allows for tracking of surgical instruments in this scene with no external tracking system necessary.

Acts 18-24 represent one embodiment for identifying the features in the coordinate system of the camera. Additional, different, or fewer acts may be used. For example, the imaging of the surgical tool uses the camera or captured images to reconstruct the model without separately acquiring depth measurements. In another example, the 3D surface is determined and a classifier identifies the features in the 3D surface.

In act 18, depth measurements are acquired. The depth measurements are acquired from an endoscope or laparoscope. The intraoperative camera is used to acquire the depth measurements, such as using stereo vision or imaging distortion on the surface from transmission of structured light (e.g., light in a grid pattern). The intraoperative endoscopic or laparoscopic images are captured with a camera-projector system or stereo camera system. In other embodiments, the depth measurements are performed by a separate time-of-flight (e.g., ultrasound), laser, or other sensor positioned on the intraoperative probe with the camera.

With the camera and/or sensor inserted in the patient, the depth measurements for measuring relative position of features, organs, anatomy, or other instruments are performed. As intraoperative video sequences are acquired or as part of acquiring the video sequences, the depth measurements are acquired. The depth of various points (e.g., pixels or multiple pixel regions) from the camera are measured, resulting in 2D visual information and 2.5D depth information. A point cloud for a given image capture is measured. By repeating the capture as the patient and/or camera move, a stream of depth measures is provided. The 2.5D stream provides geometric information about the object surface and/or other objects.

In act 20, a three-dimensional distribution of the depth measurements is created. The relative locations of the points defined by the depth measurements are determined. Over time, a model of the interior of the patient is created from the depth measurements. In one embodiment, the video stream or images and corresponding depth measures for the images are used to create a 3D surface model. The processor stiches the measurements using structure from motion or simultaneous localization and mapping. These processes deal with noise and/or inaccuracy to estimate the representation of the patient in 3D from the video and depth measurements. Other processes may be used.

The model or volume data from the camera may represent the features, but is not labeled. Features may be labeled by applying one or more classifiers to the data. Alternatively or additionally, acts 22 and 24 are performed for interactive labeling.

In act 22, a medical instrument is imaged with the video camera. The medical instrument is a surgical tool or other tool for use within the patient. The medical instrument is for surgical use, such as a scalpel, ablation electrode, scissors, needle, suture device, or other tool. Alternatively, the medical instrument is for guiding other instruments, a catheter, a probe, or a pointer specifically for use in act 24 or for other uses.

Part of the instrument, such as the tip, is positioned within the patient to be visible to or captured in images by the camera. The processor tracks the medical instrument in the video or images over time, and thus tracks the medical instrument relative to the 3D model created from the depth measurements and/or images. For example, the tip of the medical instrument is tracked in the video and in relation to the depth measurements.

The tracking determines the location or locations in three-dimensions of the tip or other part of the instrument. In one embodiment, a classifier determines the pixel or pixels in an image representing the tip and the depth measurements for that pixel or pixels indicate the location in three-dimensions. As the instrument moves, the location of the tip in three-dimensions is repetitively determined or the location is determined at triggered times.

In one embodiment, the medical instrument is segmented in one or more images from the camera (e.g., in video images from an endoscope or laparoscope). The segmentation separates the instrument from the background in an image. In other embodiments, the segmentation uses the 3D model from the depth measurements, which include points from the instrument. The instrument model or a depth pattern specific to the instrument is used to segment the instrument in the depth measurements.

Any segmentation may be used, such as fitting a statistical or other model of the instrument in the image or model or such as detecting a discriminative color and/or shape pattern on the instrument. Intensity level or color threshold may be used. The threshold level is selected to isolate the instrument, such as associated with greater x-ray absorption. A connected component analysis or low pass filtering may be performed. The largest connected region from the pixels remaining after the thresholding is located. The area associated with groups of pixels all connected to each other is determined. The largest area is the instrument. Other processes may be used, such as identifying shapes or directional filtering. In one embodiment, a machine-trained detector is applied to detect and segment the instrument. Machine training may be used to train a detector to deal with the likely scenario, such as training a detector in instrument detection in a given application. Any machine learning may be used, such as a neural network, Bayesian classifier, or probabilistic boosting tree. Cascaded and/or hierarchal arrangements may be used. Any discriminative input features may be provided, such as Haar wavelets or steerable features.

The segmentation results in locations of the instrument, such as the tip of the instrument, being known relative to the coordinate system of the camera. The instrument is tracked. FIG. 2 shows a tool positioned in the field of view of the camera at (d). The motion or change in position, such as associated with swabbing (e.g., rubbing or back and forth movement) or other pattern of motion, may be determined.

By placing the tool adjacent to, on, or other position relative to a feature in the patient, the location of the feature in the 3D model or camera coordinate system is determined. The surgical instrument is handled manually or with robotic assistance during feature digitization to indicate features.

In act 24, an indication of a feature is received. Indications of different features may be received as the medical instrument is moved or placed to point out the different features. The processor receives the indications based on the tracked position of part of the medical instrument. For example, the tip is positioned against a feature and a swabbing or other motion pattern applied. The motion of the instrument and position is detected, indicating that the swabbed surface is a feature. Alternatively, the instrument is positioned on or against the feature without motion at the feature.

The user indicates the feature based on a user interface request to identify a specific feature or by selecting the label for the feature from a menu after indicating the location. In one approach, the user places the instrument relative to the feature and then activates feature assignment, such as selecting the feature from a drop down list and confirming that the location of the tip or part of the instrument is on, adjacent, to or otherwise located relative to the feature. Based on the user input (selection or tool motion), the feature location relative to the 3D model is determined.

With the ability to track the position of an instrument tip in 3D space in the video coordinate system, the tool is used to localize anatomical salient features as points or distinctive surface patches. The instrument may be used to define the spatial extent of the feature, such as tracing a surface patch with the instrument, drawing a line or curve feature with the instrument, or designating a point with the instrument. Alternatively, the instrument is used to show the general location of the feature, but a feature model (e.g., statistical shape model for the feature) is fit to the 3D model for a more refined location determination.

The anatomical features located in the 3D model or camera coordinate system correspond to the set of features annotated in the statistical atlas or otherwise identified in the scan data. In alternative embodiments, one or more features located in the scan data are not located in the 3D model, or vise versa.

To assist in designating the features using the instrument, any previously assigned or already completed feature locations are annotated by the processor. The annotation may be text, color, texture, or other indication. The annotation may assist during navigation for refined registration and/or may handle challenging scenarios with occluded or complex structures as the features. Alternatively, annotations are not displayed to the user.

In act 26, the processor registers coordinates systems of the medical scan data from the medical scanner with the intraoperative camera using the identified features. The salient features are used to register. Rather than using a tracking sensor external to the patient, the features are used to align the coordinate systems or transform one coordinate system to the other. In alternative embodiments, an external tracking sensor is also used.

Correspondence between salient anatomical features in each image modality guides the registration process. For example, the three-dimensional distribution from the camera is registered with the preoperative volume using the salient features of the three-dimensional distribution and the salient features of the preoperative volume. The 3D point cloud reconstructed from the intraoperative video data is registered to the preoperative image volume using the salient features. The feature correspondences in the two sets of data are used to calculate registration between video and medical imaging.

Any registration may be used, such as a rigid or non-rigid registration. In one embodiment, a rigid, surface-based registration is used in act 28. The features are surface patches, so the rotation, translation, and/or scale that results in the greatest similarity between the sets of features from 3D model and the scan data is found. Different rotations, translations, and/or scales of one set of features relative to the other set of features are tested and the amount of similarity for each variation is determined. Any measure of similarity may be used. For example, an amount of correlation is calculated. As another example, a minimum sum of absolute differences is calculated.

In another embodiment, the processor rigidly registers the salient features in the medical scan data with the salient features in a three-dimensional model from the video images with a weighted surface-matching scheme. Points, line, or other features shapes may be used instead or as well. The comparison or level of similarity is weighted. For example, some aspects of the data are weighted more or less heavily relative to others. One or more locations or features may be deemed more reliable indicators matching, so the difference, data, or other aspect of similarity is weighted more heavily compared to other locations. In saliency-based global matching, more features that are salient are identified. The locations of the more salient features are weighted more heavily.

One approach for surface-based rigid registration is the common iterative closest point (ICP) registration. Any variant of ICP may be used. Different variants use different weighting criteria. The salient features are used as a weighting factor to force the registration toward a solution that favors the alignment of the features rather than the entire organ surface, which may have undergone bulk deformation. The surfaces represented in the data that are not identified features may still be used for registration or are not. Other approaches than ICP may be used for matching surfaces or intensity distributions.

FIG. 2 shows an example of registration. The patient-specific features from the atlas fitted to scan data of (c) are registered with the features in the video coordinate system from interactive features selection using the tool of (e) in the weighted registration of (f).

The registration may be handled progressively. A single surface, single curve, two lines, or three points may be used to rigidly register. Since the features in the video camera coordinate system use interaction of the instrument with each feature, the registration may be performed once the minimum number of features is located. As additional features are located, the imaging of act 22, receipt of indication of act 24 and registering of act 26 are performed again or repeated. The repetition continues until all features are identified and/or until a metric or measure of sufficient registration is met. Any metric may be used, such as a maximal allowed deviation across features (e.g., across landmarks or annotated locations). Alternatively, all of the features are identified before performing the registration just once.

The rigid registration is used for imaging or other purposes. In another embodiment, further registration is performed. The rigid registration of act 28 is an initial registration, followed by a non-rigid registration of act 30. The non-rigid registration uses residual distances from the rigid registering as partial boundary conditions. The residual distances are minimized, so are bounded to not be greater. The non-rigid alignment refines the initial rigid alignment.

Any non-rigid registration may be used. For example, the residuals themselves are the non-rigid transformation. As another example, cost functions, such as an elastic or spring-based function, are used to limit the relative displacement of a location and/or relative to other locations.

In act 32, an image of the patient is generated from the scan data and the image capture from the video camera. The 3D model from the depth measurements may be represented in the image or not. The image includes information from both coordinate systems, but using the transform resulting from the registration to place the information in a common coordinate system or to relate the coordinate systems. For example, a three-dimensional rendering is performed from preoperative or other scan data. As an overlay after rendering or combination of data prior to rendering, a model of the instrument as detected by the video is added to the image. Rather than the instrument model, an image capture from the video camera is used in the rendering as texture. Another possibility includes adding color from the video to the rendering from the scan data.

In one embodiment, a visual trajectory of the medical instrument is provided in a rendering of the preoperative volume. By using an online 3D stitching procedure, the pose of the surgical instrument is projected into a common coordinate system and may thus be used to generate a visual trajectory together with preoperative data.

In other approaches, the image may include adjacent but separate visual representations of information from the different coordinate systems. The registration is used for pose and/or to relate spatial positions, rotation, and/or scale between the adjacent representations. For example, the scan data is rendered to an image from a view direction. The video, instrument, and/or 3D model is likewise presented from a same perspective, but not overlaid.

The image is displayed. The image is displayed on a display of a medical scanner. Alternatively, the image is displayed on a workstation, computer, or other device. The image may be stored in and recalled from a PACS memory.

FIG. 3 shows one embodiment of a system for registration. The system registers a coordinate system for the medical imager 48 with a coordinate system for an endoscope or laparoscope with the camera 40. Data from the medical imager 48 is registered with images or information from the camera 40.

The system implements the method of FIG. 1. Alternatively or additionally, the system implements the method of FIG. 2. Other methods or acts may be implemented.

The system includes a camera 40, a depth sensor 42, a surgical tool 44, a medical imager 48, a memory 52, a processor 50, and a display 54. Additional, different, or fewer components may be provided. For example, a separate depth sensor 42 is not provided where the camera captures depth information. As another example, a light source, such as a structured light source, is provided on the endoscope or laparoscope. In another example, a network or network connection is provided, such as for networking with a medical imaging network or data archival system. In another example, a user interface is provided for interacting with the processor, intraoperative camera 40, and/or the surgical tool 44.

The processor 50, memory 52, and/or display 54 are part of the medical imager 48. Alternatively, the processor 50, memory 52, and/or display 54 are part of an archival and/or image processing system, such as associated with a medical records database workstation or server. In other embodiments, the processor 50, memory 52, and display 54 are a personal computer, such as desktop or laptop, a workstation, a server, a network, or combinations thereof. The processor 50, display 54, and memory 52 may be provided without other components for acquiring data by scanning a patient (e.g., without the medical imager 48).

The medical imager 48 is a medical diagnostic imaging system. Ultrasound, CT, x-ray, fluoroscopy, PET, SPECT, and/or MR systems may be used. The medical imager 48 may include a transmitter and includes a detector for scanning or receiving data representative of the interior of the patient.

The intraoperative camera 40 is a video camera, such as a charge-coupled device. The camera 40 captures images from within a patient. The camera 40 is on an endoscope, laparoscope, catheter, or other device for insertion within the body. In alternative embodiments, the camera 40 is positioned outside the patient and a lens and optical guide are within the patient for transmitting to the camera. A light source is also provided for lighting for the image capture.

The sensor 42 is a time-of-flight sensor. In one embodiment, the sensor 42 is separate from the camera 40, such as being an ultrasound or other sensor for detecting depth relative to the lens or camera 40. The sensor 42 is positioned adjacent to the camera 40, such as against the camera 40, but may be at other known relative positions. In other embodiments, the sensor 42 is part of the camera 40. The camera 40 is a time-of-flight camera, such as a LIDAR device using a steered laser or structured light. The sensor 42 is positioned within the patient during minimally invasive surgery.

The minimally invasive surgical tool 44 is any device used during minimally invasive surgery, such as scissors, clamp, scalpel, ablation electrode, light, needle, suture device, and/or cauterizer. The surgical tool 44 is thin and long to be inserted into the patient through a hole. Robotics or control wires control the bend, joints, and/or operation while inserted. The control may be manual, semi-automatic, or automatic.

The memory 52 is a graphics processing memory, a video random access memory, a random access memory, system memory, cache memory, hard drive, optical media, magnetic media, flash drive, buffer, database, combinations thereof, or other now known or later developed memory device for storing data representing anatomy, atlas, features, images, video, 3D model, depth measurements, and/or other information. The memory 52 is part of the medical imager 48, part of a computer associated with the processor 50, part of a database, part of another system, a picture archival memory, or a standalone device.

The memory 52 stores data representing labeled anatomy of the patient. For example, data from the medical imager 48 is stored. The data is in a scan format or reconstructed to a volume or three-dimensional grid format. After any feature detection and/or fitting an atlas with labeled features to the data, the memory 52 stores the data with voxels or locations labeled as belonging to one or more features. Some of the data is labeled as representing specific parts of the anatomy.

The memory 52 may store other information used in the registration. For example, video, depth measurements, a 3D model from the video camera 40, surgical tool models, and/or segmented surgical tool information are stored. The processor 50 may use the memory to temporarily store information during performance of the method of FIG. 1 or 2.

The memory 52 or other memory is alternatively or additionally a non-transitory computer readable storage medium storing data representing instructions executable by the programmed processor 50 for identifying salient features and/or registering. The instructions for implementing the processes, methods and/or techniques discussed herein are provided on non-transitory computer-readable storage media or memories, such as a cache, buffer, RAM, removable media, hard drive, or other computer readable storage media. Non-transitory computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone, or in combination. Likewise, processing strategies may include multiprocessing, multitasking, parallel processing, and the like.

In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system.

The processor 50 is a general processor, central processing unit, control processor, graphics processor, digital signal processor, three-dimensional rendering processor, image processor, application specific integrated circuit, field programmable gate array, digital circuit, analog circuit, combinations thereof, or other now known or later developed device for identifying salient features and/or registering features to transform a coordinate system. The processor 50 is a single device or multiple devices operating in serial, parallel, or separately. The processor 50 may be a main processor of a computer, such as a laptop or desktop computer, or may be a processor for handling some tasks in a larger system, such as in the medical imager 48. The processor 50 is configured by instructions, firmware, design, hardware, and/or software to perform the acts discussed herein.

The processor 50 is configured to locate anatomical positions in the data from the medical imager 48. Where the medical imager 48 provides the salient features, the processor 50 locates by loading the data as labeled. Alternatively, the processor 50 fits a labeled atlas to the data from the medical imager 48 or applies detectors to locate the features for a given patient.

The processor 50 is configured to locate anatomical positions using the surgical tool 44 represented in the images. A 3D model of the interior of the patient is generated, such as using time-of-flight to create a 3D point cloud with the sensor 42 and/or from images from the camera 40. Depth measurements for images are used to generate the 3D model in the coordinate system of the camera 40.

The processor 50 locates the anatomical positons relative to the 3D model using the surgical tool 44. The surgical tool 44 is detected in the images and/or point cloud. By isolating the location of part of the surgical tool 44 relative to anatomy in the patient, the processor 50 labels locations in the 3D model as belonging to a given feature. The surgical tool 44 is placed to indicate the location of a given salient feature. The processor 50 uses the tool segmentation to find the locations of the anatomical feature represented in the 3D model.

The processor 50 is configured to register the images with the data using the labeled anatomy and the anatomical positions. A transform to align the coordinate systems of the medical imager 48 and the camera 40 is calculated. ICP, correlation, minimum sum of absolute differences, or other measure of similarity or solution for registration is used to find the translation, rotation, and/or scale that align the salient features in the two coordinate systems. Rigid, non-rigid, or rigid and non-rigid registration may be used.

The display 54 is a monitor, LCD, projector, plasma display, CRT, printer, or other now known or later developed devise for outputting visual information. The display 54 receives images, graphics, text, quantities, or other information from the processor 50, memory 52, or medical imager 48.

One or more medical images are displayed. The images use the registration, such as a rendering form the data of the medical imager with a model of the surgical tool 44 as detected by the camera 40 overlaid or included in the rendering.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A method for registration of a video camera with a preoperative volume, the method comprising:
fitting an atlas labeled with first salient features to the preoperative volume of a patient;
acquiring depth measurements from an endoscope or laparoscope having the video camera and inserted within the patient;
imaging a medical instrument in the patient with the video camera;
receiving indications of second salient features by the medical instrument being positioned relative to the second salient features;
creating a three-dimensional distribution of the depth measurements labeled with the second salient features;
registering the three-dimensional distribution with the preoperative volume using the second salient features of the three-dimensional distribution and the first salient features of the preoperative volume; and
generating an image of the patient from the preoperative volume and a capture from the video camera, the image being based on the registering of the preoperative volume with the three-dimensional distribution.

2. The method of claim 1 wherein fitting comprises fitting, by a processor, the atlas as a statistical atlas non-rigidly with the preoperative volume.

3. The method of claim 1 wherein acquiring the depth measurements comprises acquiring with a time-of-flight sensor.

4. The method of claim 1 wherein acquiring the depth measurements comprises acquiring with the video camera in a stereo view or from a projection of structured light.

5. The method of claim 1 wherein imaging the medical instrument comprises tracking a tip of the medical instrument in video from the video camera and in relation to the depth measurements.

6. The method of claim 1 wherein receiving the indications comprises receiving user input when a tip of the medical instrument is positioned against the second salient features.

7. The method of claim 1 wherein receiving indications comprises receiving indications of points, surface patches, or points and surface patches.

8. The method of claim 1 wherein creating comprises stitching a video stream from the video camera into the three-dimensional distribution with structure from motion or simultaneous localization and mapping.

9. The method of claim 1 wherein the first and second salient features comprise surfaces and wherein registering comprises a rigid, surface-based registration.

10. The method of claim 9 wherein the rigid, surface-base registration comprises common iterative closest point registration.

11. The method of claim 1 further comprising:
performing non-rigid registration after the registering, the non-rigid registration using residual distances from the registering as partial boundary conditions.

12. The method of claim 1 wherein generating the image comprises generating the image as a three-dimensional rendering of the preoperative volume including a model of the medical instrument positioned based on the registering.

13. The method of claim 1 wherein generating the image comprises generating a visual trajectory of the medical instrument in a rendering of the preoperative volume.

14. The method of claim 1 further comprising repeating the imaging, receiving, and registering with additional second salient features until a metric in the registration is satisfied.

15. In a non-transitory computer readable storage medium having stored therein data representing instructions executable by a programmed processor for registration with medical scan data, the storage medium comprising instructions for:
identifying salient features in the medical scan data representing a patient, the medical scan data being from a medical scanner;
identifying the salient features in video images from an intraoperative camera and positioning of a tool within the patient; and
registering coordinates systems of the medical scan data from the medical scanner with the intraoperative camera using the identified salient features.

16. The non-transitory computer readable storage medium of claim 15 wherein the registering is performed with tracking of the tool without a tracking sensor external to the patient.

17. The non-transitory computer readable storage medium of claim 15 wherein identifying in the medical scan data comprises fitting a statistical model to the medical scan data, the statistical model including the salient features, wherein identifying in the video images comprises segmenting the tool in the video images and placing the tool adjacent to the salient features in the patient, and wherein registering comprises rigidly registering the salient features in the medical scan data with the salient features in a three-dimensional model from the video images.

18. A system for registration, the system comprising:
an intraoperative camera operable to captures images from within a patient;
a minimally invasive surgical tool operable to be inserted into the patient;
a memory configured to store data representing labeled anatomy of the patient, the data being from a medical imager; and
a processor configured to locate anatomical positions using the surgical tool represented in the images and to register the images with the data using the labeled anatomy and the anatomical positions.

19. The system of claim 18 wherein the processor is configured to generate a model of the patient from depth measurements for the images, the anatomical positions located relative to the model.

20. The system of claim 19 further comprising a time-of-flight sensor adjacent to the intraoperative camera.
